(19) United States
(12) Patent Application Publication
(10) Pub. No.: US 2021/0383932 A1
(43) Pub. Date: Dec. 9, 2021

(54) A DYNAMIC MULTI-FACTOR REPRESENTATION OF HEALTH DATA

(71) Applicant: University of Miami, Miami, FL (US)

(72) Inventors: Erin N. Kobetz, Miami, FL (US); Raymond R. Balise, Miami, FL (US); Zinzi Bailey, Miami, FL (US); Sheela Dominguez, Miami, FL (US); Layla Bouzoubaa, Miami, FL (US); Gustavo Abranches, Miami, FL (US); Omar Picado Roque, Miami, FL (US); Justin Stoler, Miami, FL (US); Clayton Ewing, Miami, FL (US); Gabriel Odom, Miami, FL (US)

(21) Appl. No.: 17/288,097

(22) PCT Filed: Oct. 24, 2019
(86) PCT No.: PCT/US2019/057953

§ 371 Date: Apr. 23, 2021

Related U.S. Application Data
(60) Provisional application No. 62/751,299, filed on Oct. 26, 2018.

(51) Int. Cl. G16H 50/70; G06F 16/9538; G06F 16/9537; G16H 10/60; G06T 11/20

(52) U.S. Cl. CPC G16H 50/70; G06F 16/9538; G06T 2210/41; G16H 10/60; G06T 11/206; G06F 16/9537

(57) ABSTRACT

This disclosure provides systems, methods, and computer readable media for method for displaying a multi-feature representation of health data, based on aggregated data from multiple sources. The system can include an interactive platform that can provide a multi-factor view of circumstances that drive various user-selectable health concerns in a given geographical area. The system can calculate and integrate several measures of various heath conditions, with risk factors, clinical factors, and social determinants of health on multiple levels of geography, ranging from the state to the census tract, census block, or other municipally- or privately-defined location or cell. The interactive platform can be implemented online and provide geography-based visualizations of based on multiple features including socio-demographics, disease or condition histology and staging, risk behaviors, screening behavior, environmental factors, hazardous sites, health insurance access, prevalence of potential comorbidities, housing characteristics, and residential segregation, among other features.

### CROSS-REFERENCE TO RELATED APPLICATIONS

This application claims priority to U.S. Provisional Application Ser. No. 62/751,299, filed Oct. 26, 2018, entitled “SYSTEM AND METHOD FOR ANALYZING AND DISPLAYING STATISTICAL DATA,” the contents of which are hereby incorporated by reference in their entirety.

### BACKGROUND

### Technical Field

This disclosure relates to creating and implementing a dynamically searchable database. More specifically, this disclosure is related to systems and methods for displaying a dynamic, multi-factor representation of health data, based on aggregated data from multiple sources.

### Related Art

As the second leading cause of death in the United States, cancer is a major public health problem burdening communities across the nation. However, cancer is complex, and understanding its patterning across populations involves interplay between multiple levels of factors, ranging from the biological to societal. Often statistics related to demographics, health and safety, disease, etc. are recorded and stored in completely separate datasets, and rarely, if ever, compared as complex interactions across several variables. In one example, the EPA has environmental data, the CDC has data related to behavioral risk, the census has data regarding social economics, but all are generally kept separate even though together, they have the potential to give a full view of an issue.

### SUMMARY

Systems, methods, and computer readable media for displaying a dynamic, multi-factor representation of health data, based on aggregated data from multiple sources are provided.

One aspect of the disclosure provides a computer-implemented method for displaying a dynamic, multi-feature representation of health data, based on aggregated data from multiple sources. The method can include importing, by one or more processors, data regarding a plurality of features for a plurality of census tracts to a database. The method can include defining one or more geographically defined areas as polygons and a label. The method can include overlaying the plurality of census tracts on the polygons. The method can include associating census tracts falling within a polygon to a geographically defined area defined by the polygon. The method can include performing a best fit for each census tract that crosses a boundary of the one or more geographically defined areas. The method can include associating census tracts with the one or more geographically defined areas based on the best fit. The method can include for each of the one or more geographically defined areas, aggregating the census tract data for each feature based on the associating. The method can include receiving population health data at one or more geographic levels. The method can include associating the population health data to the corresponding one or more geographically defined areas. The method can include detecting a multi-feature query of the database. The method can include generating a multi-feature visualization based on the multi-feature query.

The method can include importing data regarding a plurality of features for a plurality of census-defined places, counties, and states.

The one or more geographically defined areas can be latitude and longitude coordinates.

The polygons can be defined by points and vectors associated with specific municipally-defined areas.

The method can include defining the one or more geographically defined places or areas as a plurality of polygons based on Topologically Integrated Geographic Encoding and Referencing system (TIGER) data.

The one or more geographic levels can be one or more of a census tract, a census-defined place, a county, a collection of counties, a state, and a user-defined geography.

The population health data can be cancer data by population.

The population health data can include cancer or stroke data from at least one of the Florida Department of Health, the Florida Cancer Data System, the Florida Stroke Registry, and the Behavioral Risk Factor Surveillance System.

The population health data can be stroke data by population.

Another aspect of the disclosure provides a system for displaying a dynamic, multi-feature representation of health data, based on aggregated data from multiple sources. The system can have a database configured to store data regarding a plurality of features related to health data. The system can have one or more processors communicatively coupled to the database. The one or more processors can import data regarding a plurality of features for a plurality of census tracts to the database. The one or more processors can define a plurality of geographically defined areas as polygons with associated labels. The one or more processors can overlay the plurality of census tracts on the polygons. The one or more processors can associate census tracts falling within a polygon to a geographically defined area defined by the polygon. The one or more processors can perform a best fit for each census tract that crosses a boundary of the one or more geographically defined areas. The one or more processors can associate census tracts with the one or more geographically defined areas based on the best fit. The one or more processors can for each of the plurality of geographically defined areas, aggregate the census tract data for each feature based on the associating. The one or more processors can receive population health data at one or more geographic levels. The one or more processors can associate the population health data by geographic level to the corresponding one or more geographically defined areas. The one or more processors can receive a multi-feature query of the database. The one or more processors can generate a multi-feature visualization based on the multi-feature query.

Another aspect of the disclosure provides a computer-implemented method for displaying a dynamic, multi-feature representation of health data, based on aggregated data from multiple sources. The method can include importing, by one or more processors, data regarding a plurality of features for a plurality of municipal cells to a database. The method can include defining a plurality of geographically defined areas as polygons with labels. The method can include overlaying the plurality of municipal cells on the polygons. The method can include associating municipal cells falling within a polygon to a geographically defined area defined by the polygon. The method can include performing a best fit for each municipal cell that crosses a boundary of the plurality of geographically defined areas. The method can include associating municipal cells with the plurality of geographically defined areas based on the best fit. The method can include for each of the plurality of geographically defined areas, aggregating the municipal cell data for each feature based on the associating. The method can include receiving population health data at one or more geographic levels. The method can include associating the population health data by geographic level to the corresponding geographically defined area. The method can include detecting, by the one or more processors, a multi-feature query of the database. The method can include generating, by the one or more processors, a multi-feature visualization based on the multi-feature query.

Other features and advantages will become apparent to one of ordinary skill with a review of the following detailed description.

### BRIEF DESCRIPTION OF THE DRAWINGS

The details of embodiments of the present disclosure, both as to their structure and operation, can be gleaned in part by study of the accompanying drawings, in which like reference numerals refer to like parts, and in which:

FIG. 1 is a functional block diagram of a system for analyzing and displaying statistical data;

FIG. 2 is a flowchart of an embodiment of a method for forming a database enabling dynamic, multi-factor representation of health data;

FIG. 3 is a graphical representation of a geographically defined area used in connection with the method of FIG. 2;

FIG. 4 is a graphical representation of the geographically defined area of FIG. 3 including overlapping census tracts;

FIG. 5 is a graphical representation of a geographically defined area that overlaps multiple census tracts;

FIG. 6 is a graphical representation of a four geographically defined areas;

FIG. 7 is an example of a graphical interface for viewing age-adjusted overall cancer incidence and mortality rates in Florida, by county using the system of FIG. 1;

FIG. 8 is an example of a graphical interface for viewing age-adjusted incidence rates for cervical cancer and all cancers in Florida, by county, using the system of FIG. 1;

FIG. 9 is an example of a graphical interface for viewing age-adjusted incidence rates for cervical cancer in Florida counties, by age group, using the system of FIG. 1;

FIG. 10 is an example of a graphical interface for viewing age-adjusted incidence rates for cervical cancer among Black non-Hispanic and Hispanic women in Florida counties, using the system of FIG. 1;

FIG. 11 is an example of a graphical interface for viewing age-adjusted incidence rates for cervical cancer among non-Hispanic White women in Florida counties, using the system of FIG. 1;

FIG. 12 is an example of a graphical interface for viewing age-adjusted incidence rates for cervical cancer in Florida counties, by race/ethnicity and age group, using the system of FIG. 1;

FIG. 13 is an example of a graphical interface for viewing age-adjusted incidence rates for cervical cancer in Miami-Dade county neighborhoods, using the system of FIG. 1;

FIG. 14 is an example of a graphical interface for viewing age-adjusted incidence rates for cervical cancer in Miami-Dade county neighborhoods, zooming into the northeast quadrant of Miami-Dad County, using the system of FIG. 1;

FIG. 15 is an example of a graphical interface for viewing age-adjusted incidence rates for cervical cancer among non-Hispanic Black women in Miami neighborhoods, using the system of FIG. 1;

FIG. 16 is an example of a graphical interface for viewing age-adjusted incidence rates for cervical cancer among Hispanic women in Miami neighborhoods, using the system of FIG. 1; and

FIG. 17 is an example of a graphical interface for viewing risk and protective factors comparing the Little Haiti neighborhood to Miami-Dade county overall, using the system of FIG. 1.

### DETAILED DESCRIPTION

This disclosure presents an interactive platform that can provide a full, multi-factor view of circumstances that drive various user-selectable health concerns in a given geographical area. For example, the system can provide details regarding the cancer burden in Florida. The system can calculate and integrate several measures of, for example, the cancer burden from the Florida Cancer Data System, the state's cancer registry, with cancer risk factors, clinical factors, and social determinants of health on multiple levels of geography—ranging from the state to the census tract, census block, or other municipally- or privately-defined location or cell. The interactive platform can be implemented online and provides visualization of a variety of indicators, including socio-demographics, cancer histology and staging, risk behaviors, screening behavior, environmental factors, hazardous sites, health insurance access, prevalence of potential comorbidities, housing characteristics, and levels or degree of residential segregation, through maps and tables.

The systems and methods disclosed herein can allow the user to examine the interplay between different data sets alone and in relation to an outcome of interest, (e.g., cancer, stroke, etc.). Some mapping platforms provided by the server 101 can show the distribution of a single variable across time and place. Some allow a user to assess a representation of how the distribution of that variable is associated with a health outcome.

The systems and methods disclosed herein can allow the user to see how a variable changes in the presence of other key factors and features (for example, three or more) and ultimately how that relationship changes over time. The server 101 can provide this integration from state to neighborhood, providing compelling research, evidence-based interventions, health care delivery, and targeted recruitment efforts.

The systems and methods disclosed herein can allow a visual representation of the intersection between different features acquired from different/disparate and non-integrated datasets. In some embodiments, the data can include census geography and/or zip codes. The server 101 moves the perspective away from the traditional silo'ed approach from the perspective of a single data lens/perspective toward complex interactions across variables that have been historically measured in completely separate datasets.

For example, the influence of a superfund site on health may be exacerbated for people and places having a high level of poverty or limited education. Establishment of a mammography center can be informed by screening rates and availability of screening resources. This also ensures that insurance payers know where insured individuals live, the social and physical environment of their neighborhoods of residence, and begin planning upstream initiatives to address barriers to optimal health and healthcare utilization to reduce claims/expenses.

The systems and methods disclosed herein can help identify independent data sets that can be linked through census geography to provide a multidimensional view of health or another social phenomenon.

The systems and methods disclosed herein can further implement high level statistics to “back up” or substantiate observed relationships.

The systems and methods disclosed herein can further integrate more complex statistics to extend beyond visually observed associations to testing them.

The systems and methods disclosed herein can provide multiple measures of public health burden which mean different things (e.g., incidence versus mortality) and allow the user to see/identify how these different variables change in relation to a different outcome. This is important because the variables that drive disease onset are not the same as those that influence morbidity and/or mortality. For example, someone's smoking habits and their access to care influence cervical cancer incidence. For cervical cancer mortality, the factors of interest are different.

The systems and methods disclosed herein can integrate different data sets in a novel way providing an opportunity to identify new relationships that may merit further inquiry/exploration.

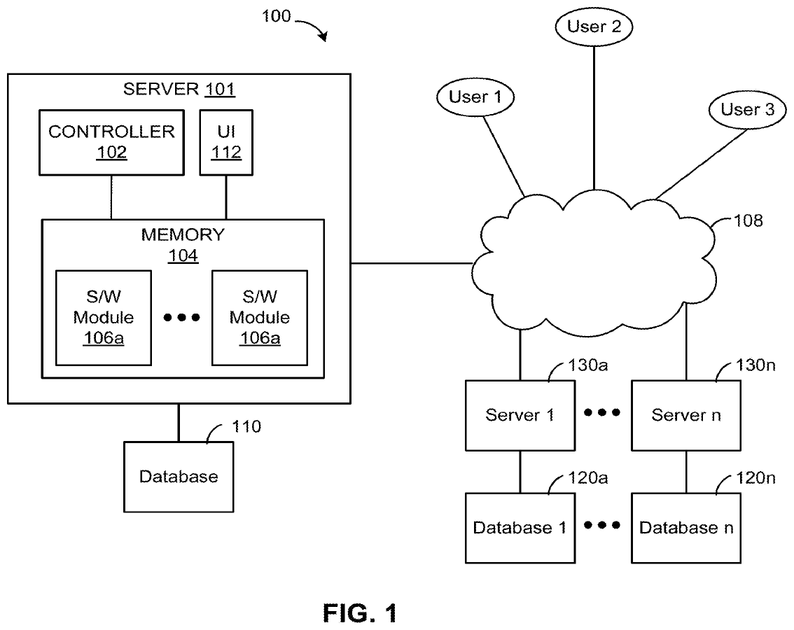

The disclosed systems, methods, and computer-readable media can provide a platform capable of displaying a dynamic, multi-factor representation of health data, based on aggregated data from multiple sources. The following description begins with an overview of various implementations of the system architecture used to realize the results captured below and described in connection with FIGS. 1-6.

Reference throughout this specification to one or more “implementations,” “one embodiment,” or “an embodiment” means that a particular feature, structure, or characteristic described in connection with the embodiment or implementation is included in at least one embodiment. Thus, appearances of the phrases “in one embodiment” or “in an embodiment” in various places throughout this specification are not necessarily all referring to the same embodiment. Furthermore, the particular features, structures, or characteristics described in connection with the “embodiments” or “implementations” may be combined in any suitable manner in one or more embodiments.

FIG. 1 is a functional block diagram of a system for analyzing and displaying statistical data. The system for analyzing and displaying statistical data (system) 100 can have a server 101. The server 101 can perform one or more of the processes disclosed herein. The server 101 can have a controller 102. The controller 102 can have a central processing unit (CPU) having one or more processors or microprocessors. In some other embodiments, the controller 102 can be a collection or group of distributed processors in a network or via cloud computing. The controller 102 can control operation of the server 101. The controller 102 may be implemented with any combination of general-purpose microprocessors, microcontrollers, digital signal processors (DSPs), field programmable gate array (FPGAs), programmable logic devices (PLDs), controllers, state machines, gated logic, discrete hardware components, dedicated hardware finite state machines, or any other suitable entities that can perform calculations or other manipulations of information.

The controller 102 may also include machine-readable media for storing software. Software shall be construed broadly to mean any type of instructions, whether referred to as software, firmware, middleware, microcode, hardware description language, or otherwise. Instructions may include code (e.g., in source code format, binary code format, executable code format, or any other suitable format of code). The instructions, when executed by the controller 102, cause the processing system to perform the various functions described herein.

The server 101 can have a memory 104 communicatively coupled to the controller 102. The memory 104 can store data and other information. The memory 104 may include both read-only memory (ROM) and random access memory (RAM), providing instructions and data to the controller 102. A portion of the memory 104 may also include non-volatile random access memory (NVRAM). The controller 102 can perform logical and arithmetic operations based on program instructions stored within the memory 104. The instructions in the memory 104 may be executable to implement the methods described herein.

The memory 104 can further have one or more software modules 106. The software modules 106 are indicated as a software module 106a through software module 106n separated by the ellipsis, indicating the presence of a plurality software modules 106. The software modules 106 can include instructions that when executed by the controller 102 perform one or more of the processes disclosed herein.

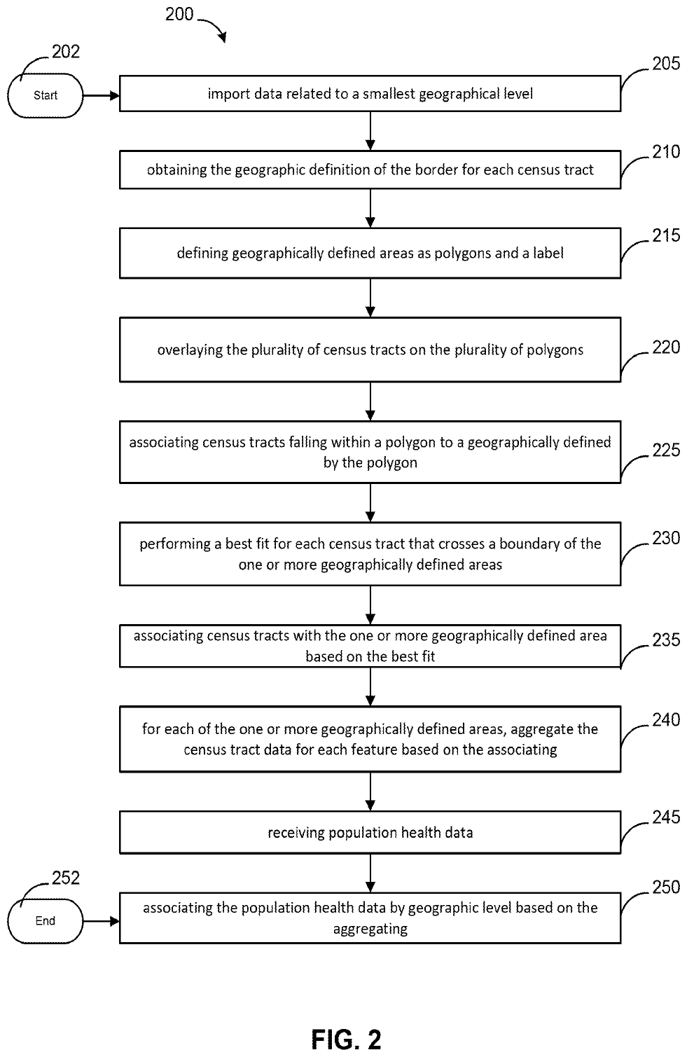

The server 101 can be coupled to a database 110. The database 110 can be populated and managed by the server 101. The database 110 can serve as a searchable repository for population health-related data that is tied to specific (e.g., predefined or user-defined) geographical areas. Formation and management of the database 110 is described in more detail in connection with FIG. 2.

In some embodiments, the server 101 can be coupled to a wide area network 108. The wide area network can include the Internet. The wide area network 108 can provide connectivity to one or more servers 130 and related databases 120. The servers 130 are shown as server 130a through server 130n , separated by the ellipsis. Any number of servers 130 is possible. The databases 120 are shown as database 120a through database 120n , separated by the ellipsis. Any number of databases 120 is possible. The databases 120 can include the various databases from which population health data is retrieved, as described below in connection with FIG. 2, for example.

The server 101 can have a graphical user interface (UI) 112. The UI 112 can be provided via, for example, the network 108. For example, one of the users of the system 100 can use a computing device having a mouse, keyboard, touchscreen, etc. to display and interact with the UI 112 provided by the server 101. Users (e.g., User 1, User 2, and User3) can access the user interface (e.g., with a home computer) to interact with the server 101 via the network 108. The server 101 can respond to queries from the user(s) and provide combined or aggregated data according to the processes disclosed herein to provide visual displays of, for example, cancer rates in comparison to various other selectable factors. As described below, the UI 112 can provide one or more pull-down menus, selection tools, and search controls for selection and analysis of one or more features.

The server 101 can import data from multiple of the databases 120 via the servers 130 and the network 108. For example, the databases 120 can include data repositories for various demographic information and health-related data in many different areas or locations. For example, the databases 120 can provide cancer or stroke data in the United States, broken down at multiple geographic levels, such as state, county, district, place, city, etc. In some examples the data can be granular to the level of census tract. In some implementations, demographic information can be included on other levels such as census block groups, census blocks, zip codes, municipalities, provinces, townships, neighborhood, and aronndissment, for example. These levels and the associated demographic information or features, can be and applicable level for use in the U.S. or other countries. This can include, for example, the American Community Survey (ACS) that provides demographic information on a census tract level. Other information on similarly granular levels is also available. The above census-defined geographies are used a primary example herein, however other minimum municipally-defined or privately-defined areas, locations, or cells can also be used, where a governing entity does not have a census, for example.

However, not all of the data are available at the same level of granularity or geographic level. The server 101 can receive or import the data from multiple databases 120 and use a common key based on geography (e.g., geographic levels) to map between the data to find common modes of comparison between the various databases 120. As used herein an exemplary “key(s)” are a set of hierarchical geographic levels. In some examples, the geographic levels can include for example, the level of the 1) state, the level of 2) collections of counties (e.g., a catchment area), the level of the 3) counties, the level of the 4) places, the level of the 5) districts within a certain area. These five geographic levels of abstraction are the primary examples used herein. However additional or user-defined/custom geographic levels may be used as needed via the user interface, for example.

In certain implementations the geographic levels or “keys” are hierarchical. For example, multiple census tracts can make up districts (5). Multiple districts can be identified in a place (4). Multiple places can be identified in a county (3). Multiple counties (3) can be identified in a collection of counties (2), and multiple counties (3) can also make up a state (1). Other keys are possible without departing from the scope of the invention. In addition, other units of geography, such as zip codes or area codes, cities, municipalities, and places can also be used as a key.

In some implementations custom geographies can be created (e.g., by a user), using census tracts or zip codes as the building blocks, and then obtaining data specific to that custom geography (e.g., block 250 of FIG. 2). Custom geography will be defined by the user, in addition to pre-defined geographies available, for example, in a drop-down menu (e.g., state, county, census-defined place, district). Census tract-level population and cancer data can be aggregated to calculate measures of cancer burden from custom geographies. In some embodiments, the controller 102 performs such calculations in real time.

The controller 102 can further perform real time statistical modeling of such data. For example, a user-defined cohort can be based on customizable parameters such as cancer types, demographic data, other social determinants, environmental, risk and protective factors in order to conduct survival analyses. The user can further specify covariates in the survival statistical model. The user can thus gain immediate access to survival models based on customizable variables that can be toggled to refine the cohort, after which a model can be exported and shared.

FIG. 2 is a flowchart of an embodiment of a method for forming a database enabling dynamic, multi-factor representation of health data. A method 200 can be used to form the database 110 in the memory 104 (FIG. 1). The method 200 can start at block 202.

At block 205 the server 101 can import data related to a smallest geographical level. As noted above, a census tract is used as a primary example of a smallest geographical level, however other implementations are possible. For example, these can include census-defined, blocks, block groups, zip codes, etc. named above, or other census-like geographies in countries other than the U.S. The census tracts, blocks, block groups, zip codes, or other census-like geographies in countries other than the U.S., can be identified by a number (e.g., numerical code) and may generally be used to tie statistics regarding the population that resides with that census tract. In that manner, statistical information regarding populations can be tied to specific locations (e.g., geographically defined areas). In areas that do not have census, the method 200 can use a smallest or minimum defined municipal cell. “Cell” in this sense can refer to a geographic location or area defined by a governing entity.

The census information can include data related to certain (demographic) features. Such features can include, but are not limited to, for example, age, race, ethnicity, native/foreign born, educational achievement, languages spoken at home, median income, percent below poverty level, rent as a percentage of income, access to a vehicle for work, percent unemployment, home ownership (and year of build), median value of owner-occupied homes, marital status, etc. These features can be reported (or recorded) on a tract-wise basis or based on other geographic levels, as needed. In some implementations the features can be summarized on any geography. The features can be variables (e.g., sociodemographic or contextual factors) that represent the combination and/or integration of census data.

In some examples, these data can be retrieved from the American Community Survey (ACS) and stored within the database 110. The ACS can provide nation-wide demographic information on a census tract level (or other census-defined geography), related to many statistics, including, for example, jobs and occupations, educational attainment, veterans, whether people own or rent their homes, etc. Sources for such information in many other regions or countries (e.g., U.S., South America, Europe, China, etc.) are also possible. The information from ACS can be retrieved on a census tract (or similar) level. Alternatively, the ACS data can be downloaded or retrieved at a census block level, or other applicable geographic level. The data pulled from ACS can include hundreds or thousands of individual census tracts. This data can later be re-conceptualized for different units or levels of geography.

In some cases, each of the features can be individually retrieved by the server 101 and stored to the database 110. The data pulled (e.g., downloaded) for each of the census tracts can be elements or puzzle pieces that can be reconfigured in order to form subsets of the data for each of the geographic levels as described below. These data can be stored (e.g., using JSON) and output for display via a web interface, for example.

In the example of ACS, the information is based upon an annual survey by the U.S. Census Bureau. The data downloaded from the ACS can include for example, the list of neighborhood details, or the above-noted features.

Data can be pulled for each feature, at one or more of the geographic levels noted above. All of the data is based initially at the level of individual census tracts and can be aggregated or arranged in subsets based on the level of the key, or geographic level in this example. Data from some databases 120 may not be available at the same level of abstraction, so the key or geographic level can be used to adapt information for viewing or comparison at a higher level of abstraction or a higher geographic level, in the present example.

At block 210, the server 101 obtains the geographic definition of the border for each census tract. This is referred to herein as a geographically defined area. In some examples, the geographically defined area can be expressed in terms of latitude and longitude (points) and vectors. The server 101 can receive geographic information defining the geographic boundaries of the census tracts. This can include associating census tracts to specific latitude and longitude (or other applicable geographic) coordinates.

In one example, the Missouri Census Data Center (MCDC) can provide such information. The MCDC provides direction as to how to assign certain census tracts to a given place. The MCDC includes data or a tool that can assign census tracks to specific geographical areas. For example, the server 101 can use the MCDC to map one geography to another geography. This can include mapping one or more census tracts, blocks, etc. to a district, city, or county, zip code or other equivalent geographical level. The MCDC shows how census tracts relate to given geographical levels.

In addition, the MCDC can provide information regarding an urban/rural distinction over a given geographic level (e.g., district, place, county, etc.). For example, the MCDC can provide data that describes how rural a portion of a given geography is. This can be a multi-level scale. For example, “Rural (<2,500,” “Urban Cluster (2,500 to <50,000),” “Urbanized Area (50,000+people).” The urban/rural distinctions are also another feature that can be stored in the database 110.

The MCDC is one example of a source of information providing geographic coordinates to the boundaries of the census tracts. Accordingly, this is not limiting on the disclosure. Other sources of such information can also be used. This can also be applied to other places outside the U.S., by identifying similar infrastructure in countries of interest.

At block 215, the controller 102 can define geographically defined areas as polygons and a label. For example, a polygon can be used to define geographic confines of a specific municipally-defined areas or locations such as a city, county, state, etc., and the label is the name associated with the geographic limits, such as the city of Miami, Miami or Miami-Dade County, or the state of Florida. In some implementations, Topologically Integrated Geographic Encoding and Referencing system (TIGER) data can be used to provide the borders (e.g., a polygon) or geospatial shapefiles for the census tracts or other census-defined areas (e.g., blocks, census block groups, census blocks, zip codes, municipalities, provinces, townships, neighborhood, and aronndissment, etc.) that match the outer boundaries of a geographically defined area. Each TIGER file can provide geospatial information related to how certain geographically defined areas (e.g., counties or cities) are drawn on a map. The TIGER file can include a complex polygon that defines the border of a county, for example. Each polygon can be geographically defined by a set of coordinates and vectors. In some examples, more than one polygon can be used to define a particular geographical area.

The TIGER files can provide tools for graphically mapping data related to the features in a visual medium/graphical representation. For example, the data associated with the codes provided with the features can be mapped to a graphical location via the TIGER data. The collection or plurality of polygons can then be provided a label (e.g., Miami). In some implementations, the each polygon can include geographical (e.g., lat/lon) coordinates and vectors describing the physical boundaries of the polygon. Cities, states, and counties, are three examples of such geographically defined areas. Other, customized or user-defined locations are also applicable.

At block 220 the controller 102 (e.g., via one or more software modules 106) can overlay the boundaries of the plurality of census tracts on the plurality of polygons. The controller 102 can then, at block 225, associate census tracts falling within a polygon to the geographically defined area defined by that polygon. Generally, only those census tracts falling completely within a polygon may be associated with that geographically defined area at block 225. For example, all of the census tracts having geographic coordinates falling within the geographic confines of the polygon that describe a city will be associated with that city, county, state, etc. (e.g., geographically defined area).

At block 230 the controller 102 can perform a best fit analysis (best fit) for each census tract that crosses a boundary of the one or more geographically defined areas. In general, many census tracts may fall on a border of a given geographically defined area. At block 230, the controller 102 can determine which tracts fall on a border of the geographically defined area (and the surrounding geographically defined areas) and perform the best fit analysis to balance population of the affected tracts and geographically define areas with the statistics associated with those features, tracts (e.g., census-defined areas), and geographically define areas.

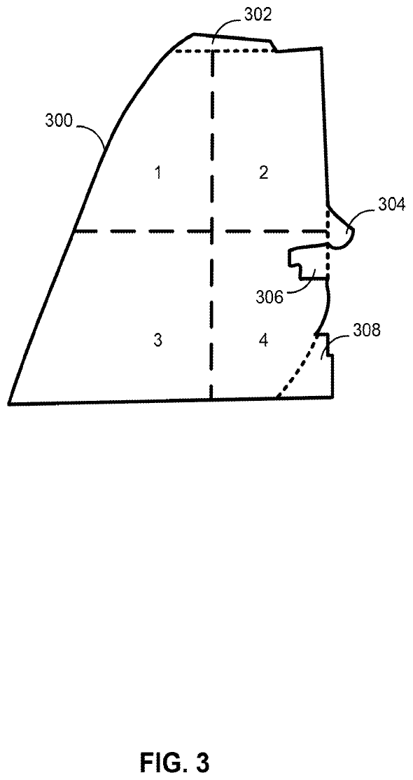

For example, a district within a city can have three census tracts that fall completely within the district, but two more census tracts that do not lie completely within the district. Ignoring the portions of the district included in the two census tracts underestimates the total population of the district, but including the additional two tracts overestimates it. The server 101 can include the census tracts received from and determine a best fit for a given geographical level. The best fit process is described more fully below in connection with FIG. 3 through FIG. 6.

At block 235 the controller 102 can associate census tracts with the one or more geographically defined areas based on the best fit. This can effectively complete the assignment of all (or nearly all; some specific examples are described below) census tracts to a geographically defined area and tie respective census tract data to one or more geographic levels based on the associated geographically defined area. In some examples, such assignment can be duplicative from one geographic level to the next. For example, a given census tract can be assigned to both City A and County B that contains City A.

At block 240 the controller 102 can, for each of the one or more geographically defined areas, aggregate the census tract data for each feature based on the associating of block 235. This process can provide aggregated information for each feature at each geographic level. For example, this step can be conceptualized as listing all of the data in a table (or multiple tables) based on geographically defined area and geographic level. In one implementation, the features can be plotted against (e.g., in rows/columns) the corresponding geographic levels.

Using the feature of “commute time” as an example, there can be a table for the selected feature (i.e., commute time), in each of state, county, place, district, tract, and/or a custom geography (e.g., the geographic levels), for each of the different states, counties, places, districts, and tracts, etc.. This can result in many (e.g., hundreds) of precalculated tables of data for each feature (e.g., stored in the database 110). There can be tables for the various units of geography (a table with state, a table with counties, a table with tracts, etc.). Each of the tables can have hundreds of records in each. In a more specific example, this could include tables for commute time (feature), for the state of Florida, each county in the state of Florida, all the places in Florida, all of the districts in Florida, and all of the tracts in Florida. This can also result in large redundancies in the saved data, allowing a calculation of rate and standard error (e.g., precision) of the data. The data may be pre-calculated or pre-aggregated and saved to the database 110 or the memory 104, for example for easy retrieval and reference.

At block 245 the server 101 can receive population health data from the servers 120. For example, various sources such as state departments of health (e.g., Florida Department of Health), Florida Cancer Data System (FCDS), the Behavioral Risk Factor Surveillance System (BRFSS), and various other databases state- and country-wide.

The FCDS, as one example, includes cancer statistics on a state-wide basis. The FCDS is a registry that includes information related to geographic, racial, and life stage information for individual instances of cancer in the state of Florida. Each of the health- or cancer-related components can be included as a feature within the database 110.

The server 101 can also retrieve information regarding other medical conditions such as strokes. The stroke-related data can also be included in the features stored within the database 110. For example, a state, local, district, or city stroke registry (e.g., the Florida Stroke Registry) can be used as a source for such health-related data.

The server 101 can, via a secure download or file transfer (e.g., FTP), download the FCDS information. FCDS provides data on each person with cancer, geocoded to their home census tract. In one example, the server 101 can calculate age-standardized cancer rates in one or more geographic areas based on the data received. These data can be stored as features within the database 110. In some embodiments, the server 101 can group census tracts as needed for a given search functions, and calculate statistics, including the age standardized cancer rates, and years of potential life lost. This can be completed based on the five or more geographic levels previously described in addition other factors including race, and life stage.

Another one of the databases 120 can be the Behavioral Risk Factor Surveillance System (BRFSS). The BRFSS is conducted by and the accumulated data is maintained by the U.S. Centers for Disease Control and Prevention. The BRFSS can include annually collected information related to different geographical areas or levels. The information collected relates to survey questions posed to individuals in different areas related to various risk factors. For example, in a first area, there may be a survey of people in a given geographical that smoke, drink a lot of soda, or receive colonoscopies after a given age. The BRFSS is a collection of useful health risk factors associated with the many chronic conditions including cancer, built over years in a given location (e.g., a county) and uses a random subset of people in that location or county. The BRFSS provides a way to characterize behavioral risk in certain subsets of people in the given location (e.g., geographical level). All of the BRFSS data (e.g., the risk factors) can be included as features stored to the database 110.

Another one of the databases 102 can be the Florida Department of Health (FDOH). The FDOH can provide information related to mortality and mortality related to cancer, for example. Mortality information can be imported based on the address of the decedent, which is then converted to census tract information based on coordinates (e.g., a latitude and longitude) of the address. The FDOH data and information can be included as features stored to the database 110.

The server 101 can further import data from multiple other databases 120. Other databases can include features from the databases 120 including different, interesting, or otherwise useful data that is geographically defined (e.g., by geographically defined area). The additional data can be retrieved and associated or otherwise overlaid or compared with the data described in connection with the foregoing features stored within the database 110. Such additional features can include, for example, the location of interesting things, such as health clinics, colonoscopy centers, mammography clinics, or other services. The additional data can include geographically-related information associated with health issues, risk or behavioral issues, and to establishments or services within different geographies.

In some implementations, the additional information (e.g., features) can include the number and location of tobacco retailers in an area, the amount of pollutants in different counties, or other similar details. Other details can include statistics and related geographical information to, for example, Residential Segregation Black/White, UV Exposure, Uninsured Children, Tobacco Retailers, Uninsured Adults, Unemployment, Some College, Premature Mortality, Physical Inactivity, Population, Percent Rural, Percent Under 18, Percent of Public Schools within 150 m of Highway, Percent Not Proficient in English, Percent Native American, Percent Near Highway, Percent Hispanic, Percent Black, Percent Asian, Long Commute, Nuclear Power Plant Exposure, Outreach Efforts 2017, Median Household Income, Foreign Born, Food Insecurity, Healthcare Costs, Limited Access to Healthy Foods, Income Inequality, High School Graduation, Drinking Water Violations 2016, Food Environment Index, Children in Poverty, Air Toxics 2011 Carbon Tetrachloride, Access to Exercise Opportunities, Air Toxics 2011 Benzene, Adult Smoking, Air Toxics 2011 Formaldehyde, Adult Obesity, Air Toxics 2011 Acetaldehyde, Air Toxics 2011 1,3 butadiene, Percent Insufficient Sleep. The foregoing list is not limiting on the disclosure. Other data and information are available for use with the system 100. All of the above examples can be stored as features in the database 110.

The server 101 can also import data from a plurality of other sources including one or more public or government databases (e.g., EPA, CDC, or a variety of county or state sources of data).

In addition, further granularity can be added to the database by including patient-level data, such as integration with Electronic Health Records (EHRs). The EHRs can each be geographically associated with a census tract via a patient address, for example. This can allow the system 100 to map aggregate patient counts on a molecular level using genetic information, for example. This can include individual patient diagnoses, demographics, laboratory values, medications, visits, hospitalizations, providers, financial class, payors, genetics/genomics, and more. Much of this information may be subject to various restrictions on use, such as HIPAA (Health Insurance Portability and Accountability Act of 1996) in the United States, and similar personally identifiable information (PII) regulations in other countries. While patient-specific information can be tied to specific census tracts, the information can also be de-identified sufficiently so as to comply with relevant regulations, such as HIPAA.

In some further implementations, the database formed using the method 200 can include integration of various augmented reality and/or virtual reality platforms allowing highly customizable visualizations of the data stored and searchable in the database.

At block 250 the controller 102 can associate the population health data by census tract based on the aggregations of block 235. The data pulled in from the various servers 120 can then be categorized and aggregated by location, all based on one or more of the geographic levels. The data can then be available for query by one or more users. The one or more of the users (FIG. 1) can use the graphical user interface to perform multi-factor or multi-feature queries on the database 110 formed by the method 200. The server 101 can then generate tabular summaries and/or visualizations of the multi-factor or multi-feature queries. The graphical user interface can visualize or display the generated visualizations or representations (e.g., tables, plots or diagrams) of multiple (two or more) data sets for a visual/graphical comparison. The following figures show exemplary plots for comparison but more are possible, as desired. In some embodiments, more than two sets of data or features can be compared and contrasted using the system 100. For example, late stage diagnosis breast cancer, mammography utilization, and the presence of American college of radiology mammography centers can be plotted simultaneously in the multi-feature visualizations.

The server 101 can implement an application program interface (API) to provide unified access to data stored in separate backend systems (depending on the categorization of the data) to the application frontend and user interface. The server 101 can store the data in, for example, MongoDB.

Support data can be stored in a SQL Server and can have items necessary to present the user interface options such as search type, location and other filtering options. Data is created and managed using Sitecore, allowing application owners to modify and add new options to the user interface as needed through the Sitecore administrative interface. Individual search filter options have numerous configuration options in the administrative interface allowing application owners to fine-tune how and where the associated datasets are retrieved and displayed.

Visualization data can be stored in MongoDB and can include all of the raw datasets and geographic data rendered by the application such as cancer rates, spatial boundaries, geocoded resources and population statistics. The custom API provides access to this data and includes support for filtering queries based on options selected in the user interface.

The method 200 can end at block 252.

FIG. 3 through FIG. 6 are graphical depictions of a portion of the method of FIG. 2.

FIG. 3 is a graphical representation of a geographically defined area used in connection with the method of FIG. 2. A geographically defined area 300, such as a village, for example, can have census tracts which fall completely within it. The solid outer line shown in FIG. 3 represents the geographically defined area 300 (e.g., geographically defined area) encompassing four exemplary census tracts (labeled 1-4). The dashed lines represent the boundaries between the four exemplary census tracts. Spaces 302, 304, 308 fall between the solid line and the dotted lines and represent areas that are not encompassed by the four census tracts that fall completely within the geographically defined area. The space 306 is where the boundary of the geographically defined area 300 falls inside census tract 4.

As noted above, a hierarchy of geographically defined levels can be used. For example, the hierarchy can range from State, to County, to Census Defined Places (e.g., city, town, village, etc.) and to Neighborhoods defined within a city. The hierarchy can be used to translate or map data between geographically defined areas.

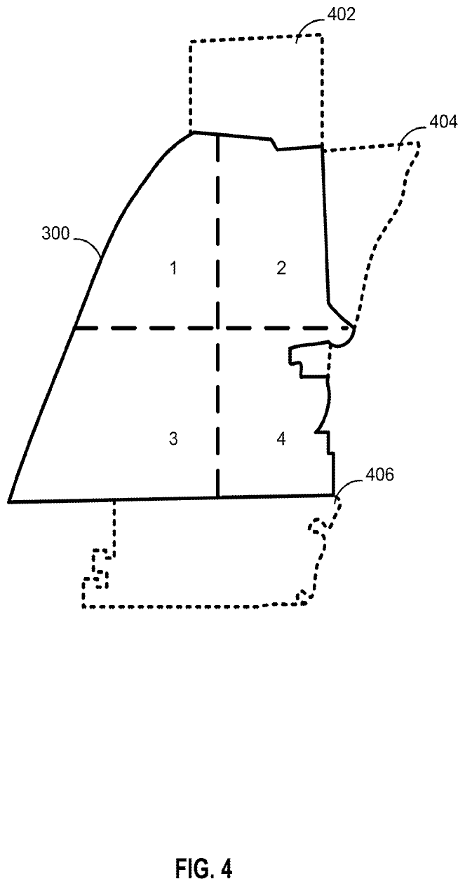

FIG. 4 is a graphical representation of the geographically defined area of FIG. 3 including overlapping census tracts. The geographically defined area 300 can overlap with census tracts 402, 404, 406. The census tracts 402, 404, 406 need to be included (assigned) with the four tracts (1-4) which fall completely within the geographically defined area 300, to obtain complete coverage of the geographically defined area 300. In this example, the three additional census tracts 402, 404, 406 intersect the geographically defined area 300. The three additional census tracts 402, 404, 406 are only partially within the geographically defined area 300.

The census tracts 402, 404, 406 that need to be included to complete the coverage are shown in dotted lines. The geographically defined area 300 can have one or more characteristics (or features) associated with it. In one example, the geographically defined area 300 is a village and the characteristic is the population of the village. Each of the census tracts shown also has a population associated with it. Including all of the census tracts that cross the boundary of a place (e.g., the geographically defined area 300) overestimates population count for the village because it includes population that is outside of the village. In one example, the total population of all of the census tracts 402, 404, 406 that cross the boundary of the geographically defined area is over 28,000. However, the population of the geographically defined area 300 is known to be 18,917 (for example from the U.S. Census Bureau's data statistics on Census Defined places). The total population of the census tracts 1-4 that fall completely within the boundary of the geographically defined area 300 is 16,986.

In an example the controller 102 can assign census tracts that intersect the boundary of more than one geographically defined area by looking to which area gets closest to its actual population by including the intersecting census tract (e.g., the census tracts 402, 404, 406), and which area contains a majority of the population of that census tract. For example, a best fit algorithm can be used as in block 230 (FIG. 2). Once the census blocks are assigned to the geographically defined area 300, the data associated with those census blocks can be associated with that geographically defined area 300. In some other implementations, the best fit process can use other refinements such as population density in smaller and smaller geographies to select a “best fit” for a given tract, or other geographic cell. Advantageously, if a tract is happens to cross multiple “place” boundaries (as depicted in FIG. 4), cancer cases, for example, in that tract will be assigned the place (e.g., geographically defined area) with the largest population. This can avoid double counting population health statistics and inserting bias into rates. Thus, certain statistics (e.g., cancer cases) from a tract that has 28k people are not associated with a place/area that only has 18k people.

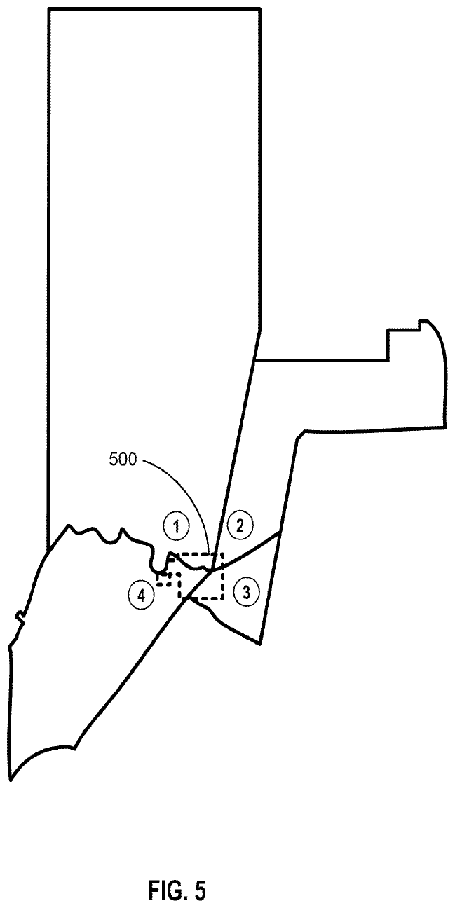

FIG. 5 is a graphical representation of a geographically defined area that overlaps multiple census tracts. A geographically defined area 500 is indicated with a dotted line and the four census tracts 1-4 (shown with solid lines) that it overlaps. The geographically defined area 500 can be, for example, a village. This represents another issue in assigning census tracts to a geographically defined area. In this example, the geographically defined area 500 has a very small population and falls within four census tracts numbered 1-4. The four census tracts have a population in the thousands. In this case, no census tract is assigned to the geographically defined area. This figure represents the problem where the population is so low for a geographically defined area that reporting certain types of information, for example, medical information, may violate the privacy (e.g., HIPAA regulations) of the residents. In some examples, this issue can be addressed by creating geographies that have a larger population than the limits imposed by HIPAA

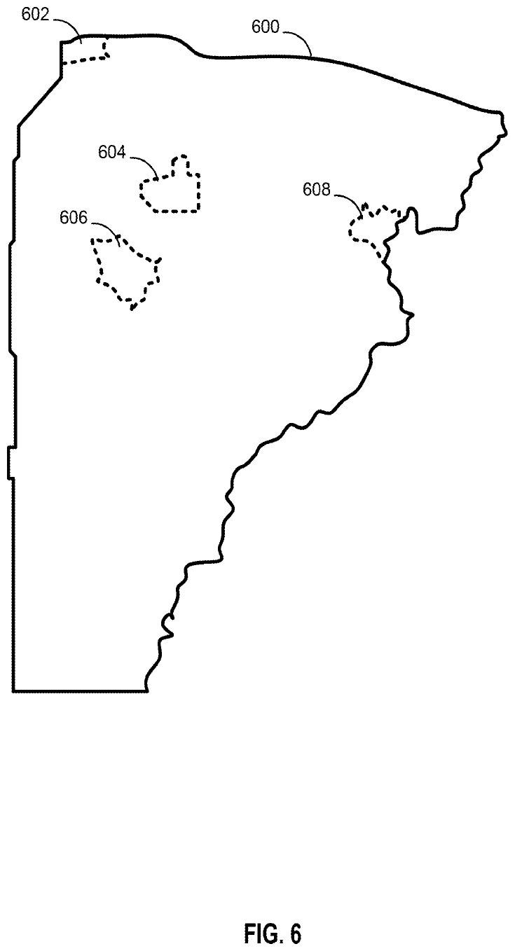

FIG. 6 is a graphical representation of a four geographically defined areas 602, 604, 606, 608 (e.g., villages) shown with dotted lines and the single census tract 600 within which all four geographically defined areas are contained. This issue is addressed by assigning the census tract 600 to one of the four areas 602, 604, 606, 608 and removing (or ignoring) the other three. In one embodiment the census tract 600 is assigned to the geographically defined area with the largest population.

The process of block 235 can include comparing the population of each of the overlapping tracts/blocks and that of the geographically defined areas 300, 500, 600 to determine how to best associate/allocate the tracts and to which geographically defined area. In some examples, no census tracts may be allocated. In other examples, as in the geographically defined area 300 (FIG. 4), the best fit may cause the tract 2 to be allocated to the geographically defined area 300 while the tract 4 may be allocated to an adjacent geographically defined area, based on the known population of the geographically defined area 300. This can, for example, allocate the tracts based on a combined population count of the combined tracts 1, 2, 3 (e.g., to remain close to of the known population of the geographically defined area 300). Tract 4 may not be allocated to the geographically defined area 300 because it would put the total population far above the total known population of the geographically defined area 300. It can then be associated or allocated to an adjacent geographically defined area. The associations made to the geographically defined area 300 can then influence the best fit for adjacent geographies. This process can be repeated on a large scale to assign all, or nearly all tracts to a given geography.

### System Functions

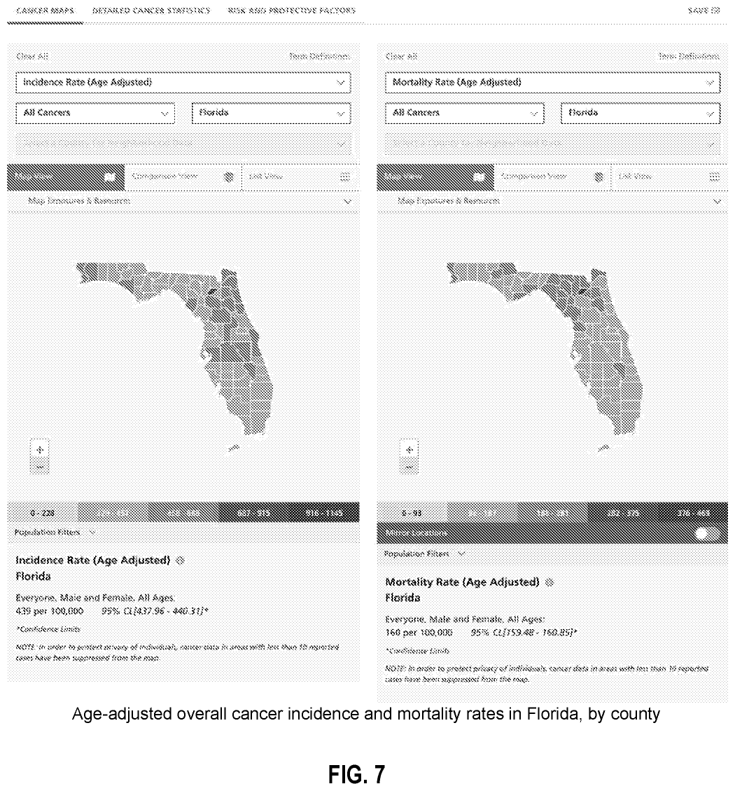

FIG. 7 is an example of a graphical interface for viewing age-adjusted overall cancer incidence and mortality rates in Florida, by county using the system of FIG. 1.

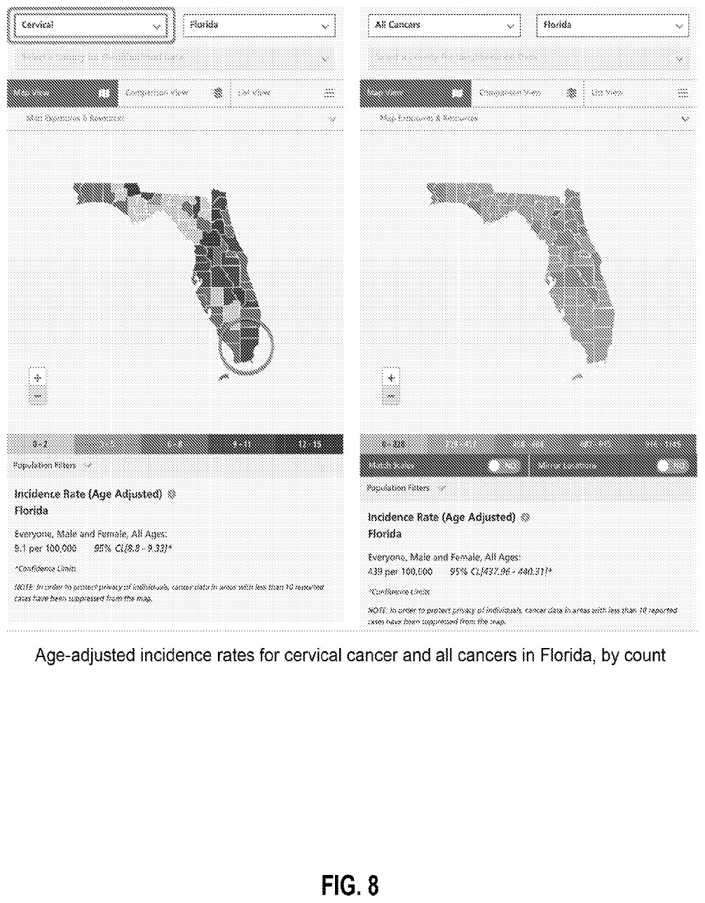

FIG. 8 is an example of a graphical interface for viewing age-adjusted incidence rates for cervical cancer and all cancers in Florida, by county, using the system of FIG. 1.

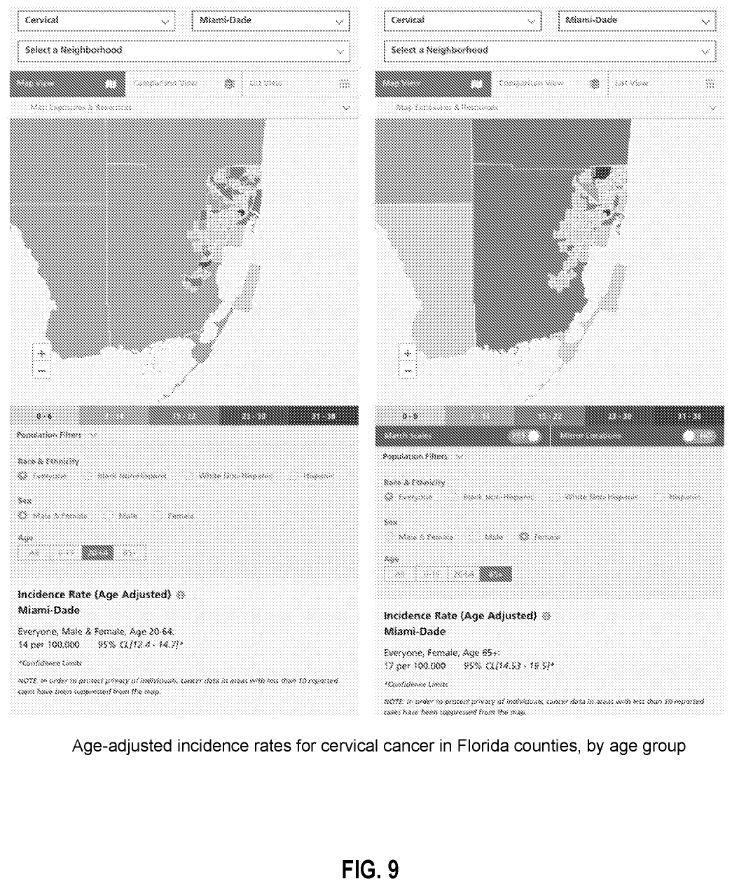

FIG. 9 is an example of a graphical interface for viewing age-adjusted incidence rates for cervical cancer in Florida counties, by age group, using the system of FIG. 1.

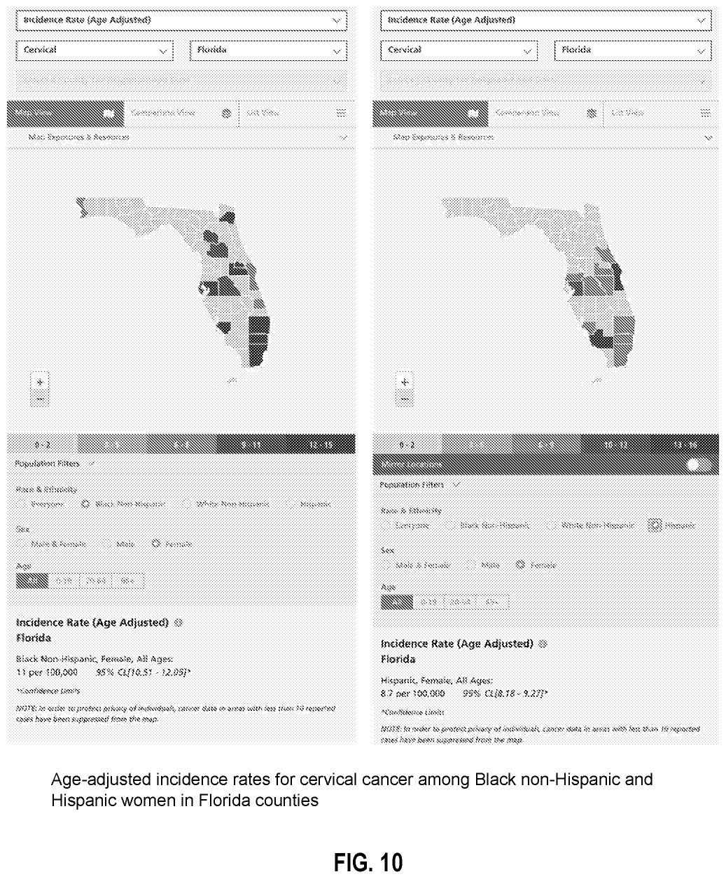

FIG. 10 is an example of a graphical interface for viewing age -adjusted incidence rates for cervical cancer among Black non-Hispanic and Hispanic women in Florida counties, using the system of FIG. 1.

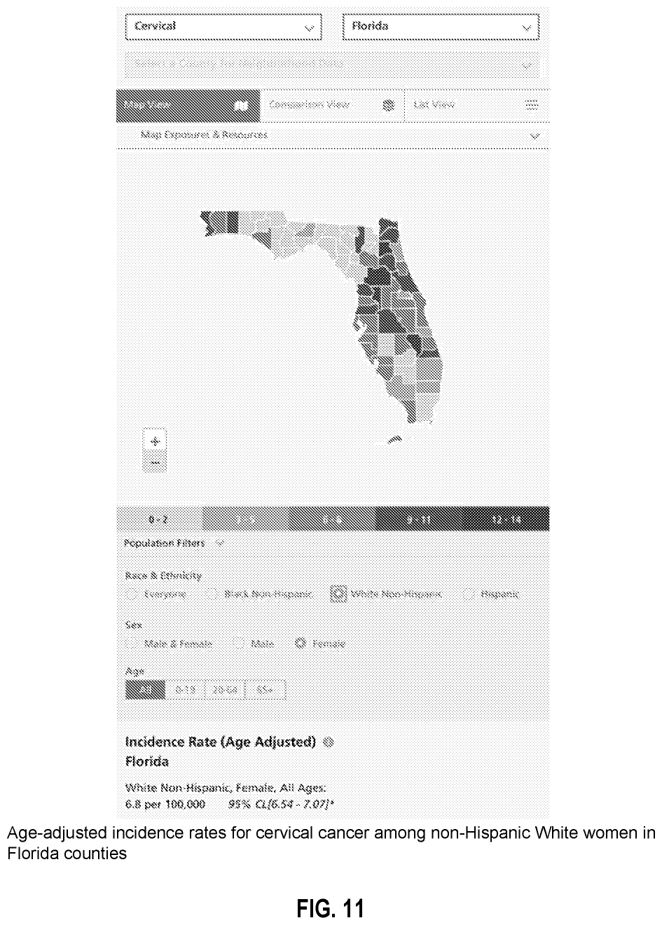

FIG. 11 is an example of a graphical interface for viewing age -adjusted incidence rates for cervical cancer among non-Hispanic White women in Florida counties, using the system of FIG. 1.

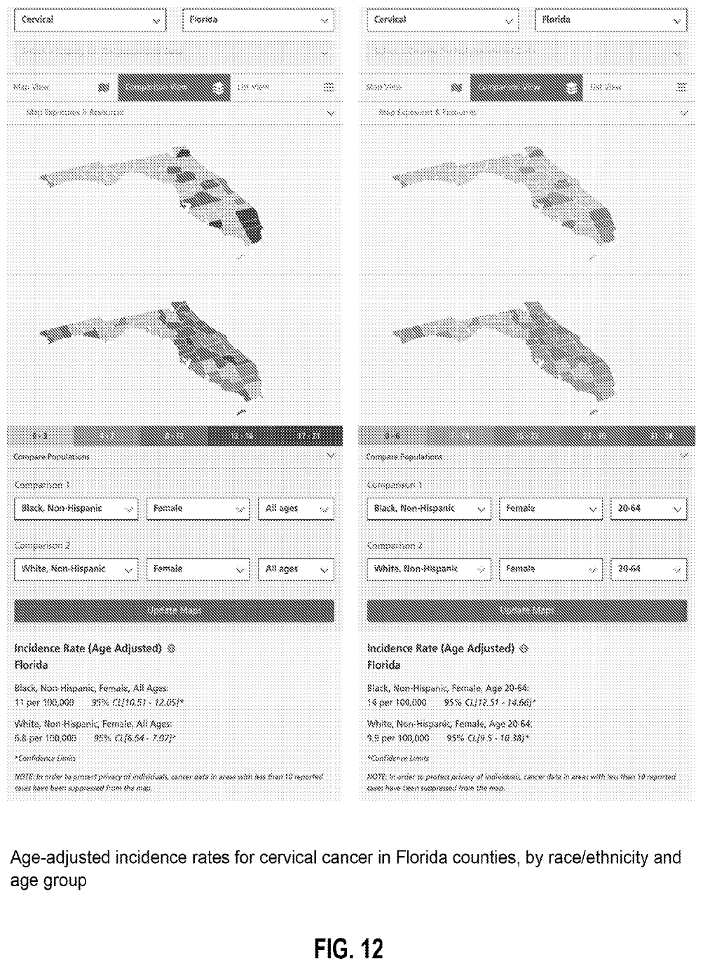

FIG. 12 is an example of a graphical interface for viewing age -adjusted incidence rates for cervical cancer in Florida counties, by race/ethnicity and age group, using the system of FIG. 1.

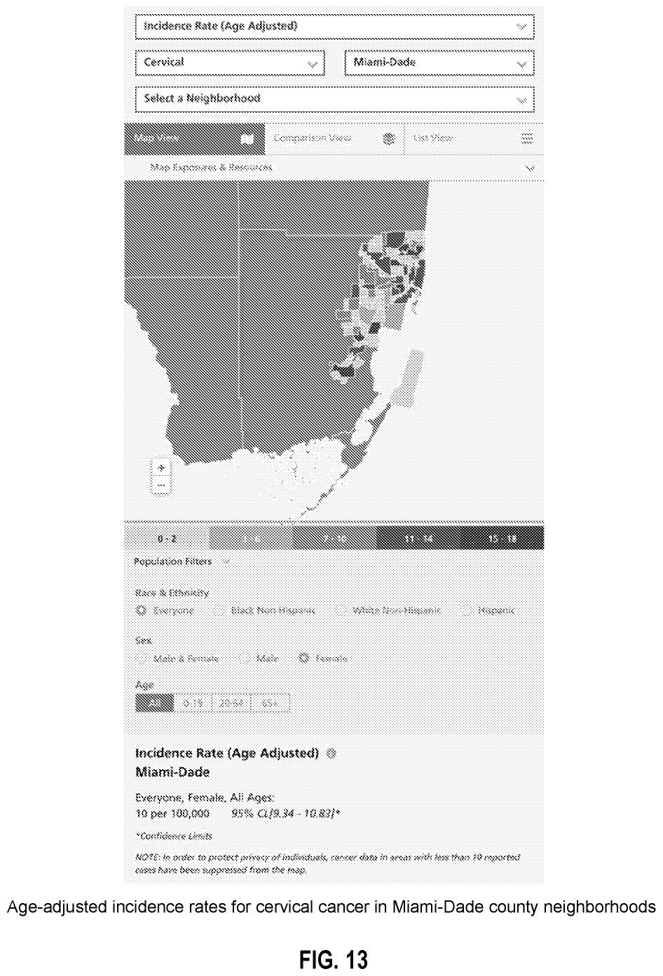

FIG. 13 is an example of a graphical interface for viewing age -adjusted incidence rates for cervical cancer in Miami-Dade county neighborhoods, using the system of FIG. 1.

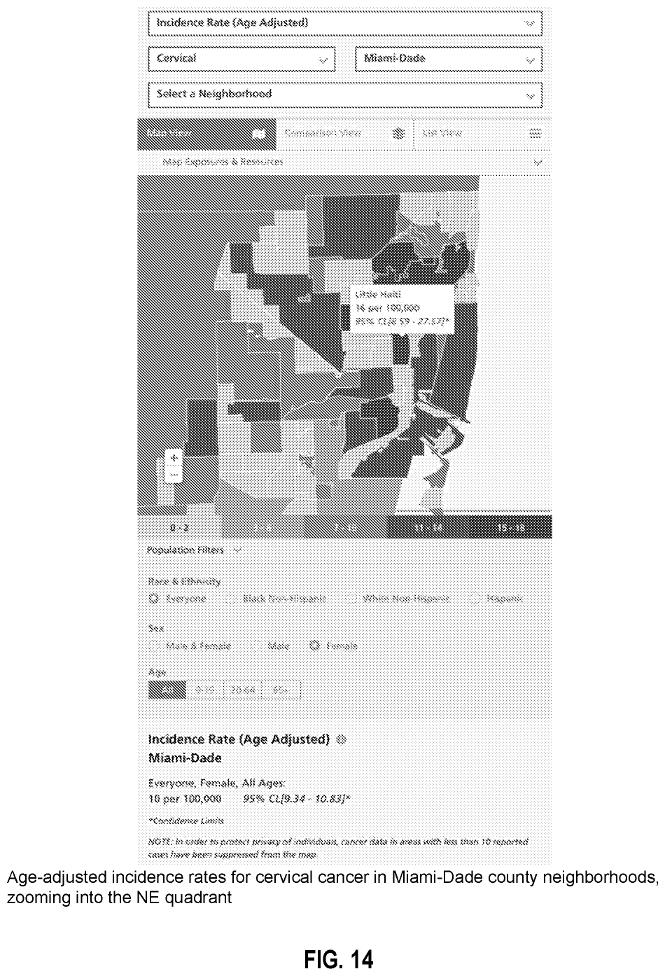

FIG. 14 is an example of a graphical interface for viewing age -adjusted incidence rates for cervical cancer in Miami-Dade county neighborhoods, zooming into the northeast quadrant of Miami-Dad County, using the system of FIG. 1.

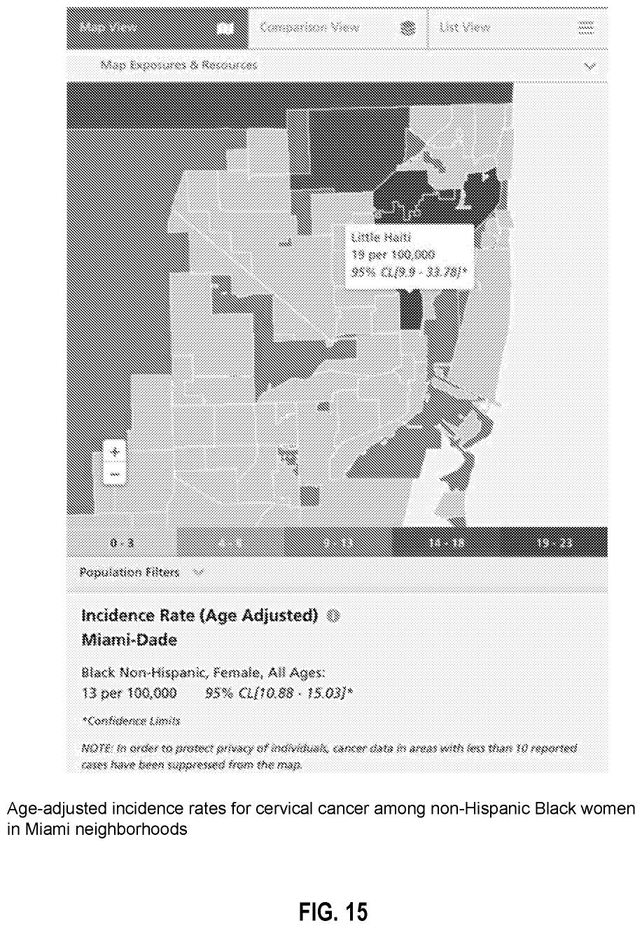

FIG. 15 is an example of a graphical interface for viewing age -adjusted incidence rates for cervical cancer among non-Hispanic Black women in Miami neighborhoods, using the system of FIG. 1.

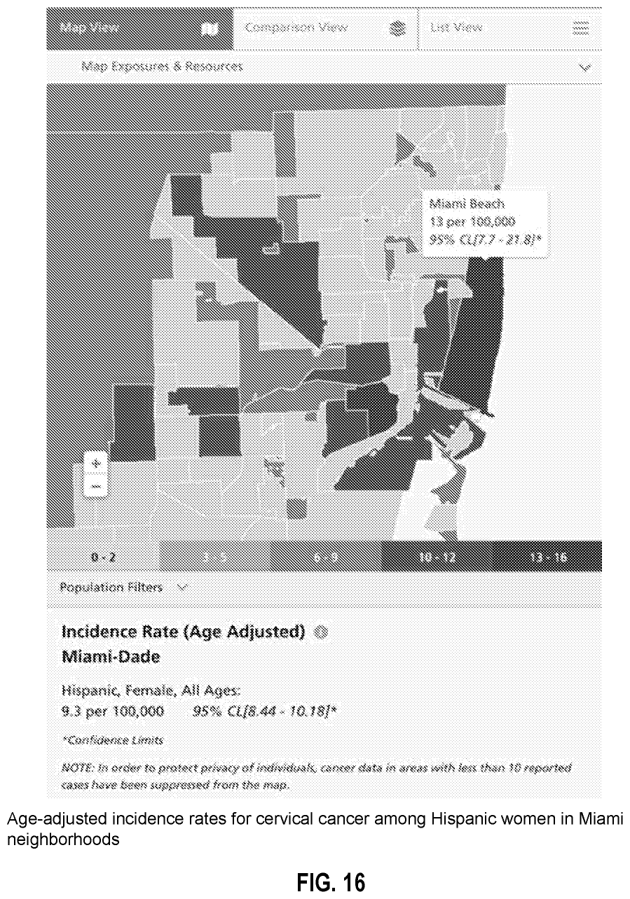

FIG. 16 is an example of a graphical interface for viewing age -adjusted incidence rates for cervical cancer among Hispanic women in Miami neighborhoods, using the system of FIG. 1.

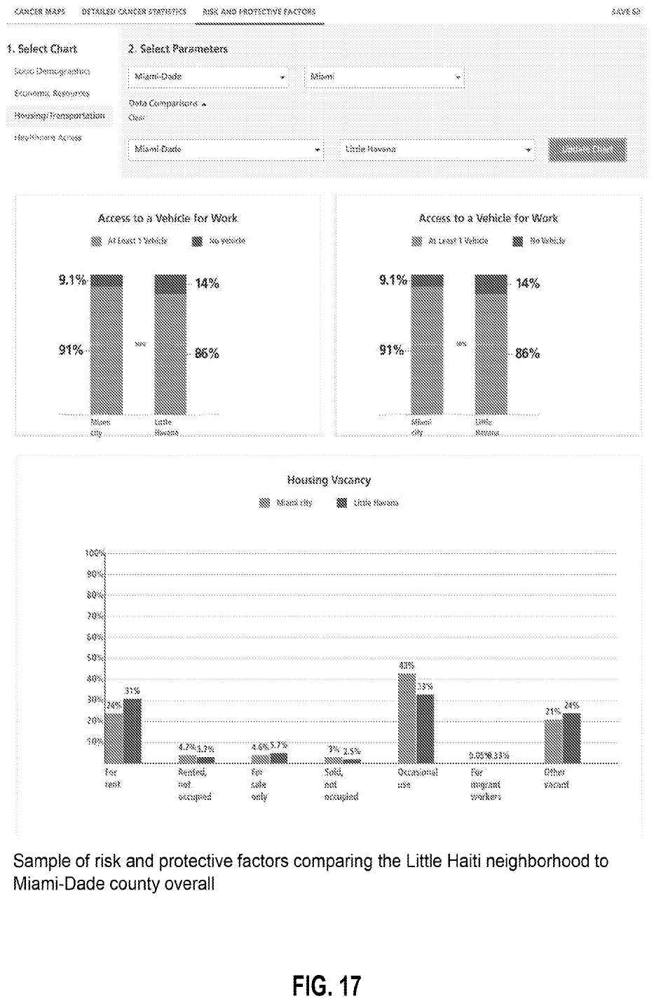

FIG. 17 is an example of a graphical interface for viewing risk and protective factors comparing the Little Haiti neighborhood to Miami-Dade county overall, using the system of FIG. 1.

The systems and methods disclosed herein can allow the user to examine the interplay between different data sets alone and in relation to an outcome of interest, (e.g., cancer). Some mapping platforms provided by the server 101 can show the distribution of a single variable across time and place. Some allow a user to assess a representation of how the distribution of that variable is associated with a health outcome.

The UI 112, for example, can provide a means for a user (e.g., the User 1, 2, 3 of FIG. 1) to selected multiple features, for example, through drop down windows as depicted in the FIG. 7 through FIG. 17. The server 101 can then display the selected features overlaid on respective geographically defined areas. Some of the feature data may not be available on all of the geographical levels. For example, “commute time” per geographically defined area may be available at the census tract level, however, “days of sunshine” may not be available on census tract level. “Days of sunshine” may be recorded per city or country and therefore can be imputed for census tracts falling within those areas. In contrast, certain features such as the “location of mammography centers” may be available by city only, and therefore may not be imputed to the census tract level. Accordingly, the geographic level at which the features from the database 110 may be compared can be a factor of the lowest common geographical level. The following description of FIG. 7 through FIG. 17 includes such selection of exemplary features via, for example, the UI 112 (FIG. 1).

Using cancer as an example, the system 100 can integrate several measures of cancer burden (features), including age-adjusted incidence, age-adjusted mortality, percent late stage diagnosis, and years of potential life lost, and integrates data from numerous sources into one user-friendly platform. This tool allows multilevel research using exported data. For example, the system 100 can provide insight into the frailty survival modeling that uses both person level and neighborhood level factors to predict a woman's hazard of death from ovarian cancer. In a first query of the system 100 looking at age-adjusted overall cancer incidence and mortality rates in Florida, by county shown in FIG. 7, the result in central and northern Florida are consistent with rural health disparities and proximity to the Deep South. However, when focusing on cancer control and prevention within the Sylvester Comprehensive Cancer Center catchment area (the four-county region of Miami-Dade, Broward, Palm Beach, and Monroe counties), there are specific cancers that disproportionately contribute to the cancer burden among individuals and communities.

For instance, focusing on cervical cancer in FIG. 8, incidence rates for Miami-Dade, Broward, and Monroe counties stand out from neighboring counties. Further, exploring the population filters, cervical cancer in Miami-Dade stands out at increasing ages, especially for women over 65 years, as shown in the side-by-side maps of FIG. 9. The age-adjusted cervical cancer incidence rate for women aged 20-64 in Miami-Dade is estimated at 14 per 100,000 (with a confidence interval that includes the statewide rate for the same age group), while the rate for women aged 65 and over in Miami-Dade is 17 per 100,000 (vs. 11 for the entire state within the same age group). This can be reflective of several varied factors, including geographic distribution of people across different ages, which can be investigated through further population filters. For example, we can also look at cervical cancer across race and ethnicity. Using the Population Filters to focus specifically on Black Non-Hispanic women, Miami-Dade, Broward, and Palm Beach stand out with the highest rates of cervical cancer (FIG. 10; map on left). To a lesser degree, we see the same counties in our catchment area highlighted when we look at Hispanic women (FIG. 10; map on right).

This is distinctive, especially in comparison with the pattern of incidence among White Non-Hispanic women in the same counties (FIG. 11). Again, this is likely to reflect many varied factors, including distribution of racial and ethnic groups (with different age distributions) across different geographies, that can be better investigated through focused study. However, looking at the magnitude and precision of the disparity in incidence, we can infer that the burden of cervical cancer (specifically, incidence) for Miami-Dade, Broward, and Palm Beach counties is concentrated among Black (and to a lesser extent, Hispanic) women in South Florida.

We can look at this in another way through the comparison view, which magnifies the ability to display geography- and population-based contrasts (FIG. 12). While contextualizing the rates within the broader landscape of Florida, documenting and visualizing this disparity on the county level is not sufficient to guide targeted outreach and research. We have to look more closely at the heterogeneity within each county. Zooming into Miami-Dade County in Map View, we can get a sense of geographic heterogeneity (FIG. 13). Choosing a county allows the user to select even smaller levels of geography—neighborhoods.

Zooming in even further, we see that neighborhoods like Little Haiti, North Miami, Model City, West Little River, Golden Glades, Homestead, Leisure City, and University Park have the highest rates of cervical cancer in the county, denoted by the darkest green shade (FIG. 14). Further, if we restrict cervical cancer incidence to Black Non-Hispanic women, we observe the highest rates in Miami Gardens (16 per 100,000), Little Haiti (19 per 100,000) and North Miami (23 per 100,000) (FIG. 15). In turn, if we restrict cervical cancer incidence to Hispanic women, we see different neighborhoods stand out, including Hialeah, Allapatah, Little Havana, Miami Beach, and Homestead, not surprisingly predominantly Hispanic/Latinx communities (FIG. 16).

The system 100 can also provide data about each neigborhood, allowing comparisons across neighborhoods with regard to environment, composition, and resources. If we compare Little Haiti to the City of Miami (the urban center of Miami-Dade County), we see that 71% of Little Haiti residents experience extreme rent burden, meaning more than 50% of their income is spent on housing (FIG. 17).

Further, we see more housing vacancy and relatively less housing dedicated to “occasional use,” likely vacation homes. Together, this snapshot may be reflective of neighborhood change occurring in Little Haiti that is less present in the City of Miami. The resources and social support in Little Haiti may be disrupted by neighborhood change and impact cancer risk, treatment, and survival. In addition to risk and protective factors, SCAN 360 affords the opportunity to delve even deeper into detailed cancer statistics, including age at diagnosis, histology, and percent late stage diagnosis.

Recognizing the multiple levels of interplay that come to bear in the patterning of health and health inequities, we can identify key areas to work in and build relationships to reduce and eventually eliminate cancer health disparities specific to our communities. The system 100 provides a platform and resources to analyze this causal interplay, and can help guide cancer control and prevention efforts. This can also help highlight areas of investigation and outreach that are particularly catchment-relevant.

The system 100 can be used to identify key areas to work in and build relationships to reduce and eventually eliminate cancer health disparities specific to our communities. The system 100 provides a platform and resources to analyze this causal interplay, and can help guide cancer control and prevention efforts. In the example of cancer centers within Florida, the system 100 can help highlight areas of investigation and outreach that are particularly catchment-relevant. For instance, the burden of cervical cancer is a particular concern for the catchment area of Sylvester Comprehensive Cancer Center, especially given the concentration of immigrant populations with limited access to HPV vaccination both in their home countries and in their current communities as well as less access to methods of secondary prevention (e.g., cervical cancer screening, HPV co-testing). Other disease sites or features may be relevant for other cancer centers in the state, allowing each to allocate resources accordingly.

### Other Aspects

The accompanying claims and their equivalents are intended to cover such forms or modifications as would fall within the scope of the disclosure. For instance, the example apparatuses, methods, and systems disclosed herein may be applied to systems, methods, and computer-readable media for selecting, overlaying, and analyzing interplay between multiple levels of features, including many different demographic, biological, health-related, and societal factors and characteristics. The various components illustrated in the figures may be implemented as, for example, but not limited to, software and/or firmware on a processor or dedicated hardware. Also, the features and attributes of the specific example embodiments disclosed above may be combined in different ways to form additional embodiments, all of which fall within the scope of the disclosure.

The foregoing method descriptions and the process flow diagrams are provided merely as illustrative examples and are not intended to require or imply that the operations of the various embodiments must be performed in the order presented. As will be appreciated by one of skill in the art the order of operations in the foregoing embodiments may be performed in any order. Words such as “thereafter,” “then,” “next,” etc. are not intended to limit the order of the operations; these words are simply used to guide the reader through the description of the methods. Further, any reference to claim elements in the singular, for example, using the articles “a,” “an,” or “the” is not to be construed as limiting the element to the singular.

The various illustrative logical blocks and algorithm operations described in connection with the embodiments disclosed herein may be implemented as electronic hardware, computer software, or combinations of both. To clearly illustrate this interchangeability of hardware and software, various illustrative components, blocks, modules, and operations have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present inventive concept.

The hardware used to implement the various illustrative logical or functional blocks described in connection with the various implementations disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of receiver devices, e.g., a combination of a DSP and a microprocessor, a plurality of microprocessors, one or more microprocessors in conjunction with a DSP core, or any other such configuration. Alternatively, some operations or methods may be performed by circuitry that is specific to a given function.

In one or more exemplary embodiments, the functions described may be implemented in hardware, software, firmware, or any combination thereof. If implemented in software, the functions may be stored as one or more instructions or code on a non-transitory computer-readable storage medium or non-transitory processor-readable storage medium. The operations of a method or algorithm disclosed herein may be embodied in processor-executable instructions that may reside on a non-transitory computer-readable or processor-readable storage medium. Non-transitory computer-readable or processor-readable storage media may be any storage media that may be accessed by a computer or a processor. By way of example but not limitation, such non-transitory computer-readable or processor-readable storage media may include random access memory (RAM), read-only memory (ROM), electrically erasable programmable read-only memory (EEPROM), FLASH memory, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory computer-readable and processor-readable media. Additionally, the operations of a method or algorithm may reside as one or any combination or set of codes and/or instructions on a non-transitory processor-readable storage medium and/or computer-readable storage medium, which may be incorporated into a computer program product.

It is understood that the specific order or hierarchy of blocks in the processes/flowcharts disclosed is an illustration of exemplary approaches. Based upon design preferences, it is understood that the specific order or hierarchy of blocks in the processes/flowcharts may be rearranged. Further, some blocks may be combined or omitted. The accompanying method claims present elements of the various blocks in a sample order, and are not meant to be limited to the specific order or hierarchy presented.

The previous description is provided to enable any person skilled in the art to practice the various aspects described herein. Various modifications to these aspects will be readily apparent to those skilled in the art, and the generic principles defined herein may be applied to other aspects.

Thus, the claims are not intended to be limited to the aspects shown herein, but is to be accorded the full scope consistent with the language claims, wherein reference to an element in the singular is not intended to mean “one and only one” unless specifically so stated, but rather “one or more.”

The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more.

Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C.

Although the present disclosure provides certain example embodiments and applications, other embodiments that are apparent to those of ordinary skill in the art, including embodiments which do not provide all of the features and advantages set forth herein, are also within the scope of this disclosure. Accordingly, the scope of the present disclosure is intended to be defined only by reference to the appended claims.

1. A computer-implemented method for displaying a dynamic, multi-feature representation of health data, based on aggregated data from multiple sources, the method comprising:
importing, by one or more processors, data regarding a plurality of features for a plurality of census tracts to a database;
defining one or more geographically defined areas as polygons and a label;
overlaying the plurality of census tracts on the polygons;
associating census tracts falling within a polygon to a geographically defined area defined by the polygon;
performing a best fit for each census tract that crosses a boundary of the one or more geographically defined areas;
associating census tracts with the one or more geographically defined areas based on the best fit;
for each of the one or more geographically defined areas, aggregating the census tract data for each feature based on the associating;
receiving population health data at one or more geographic levels;
associating the population health data to the corresponding one or more geographically defined areas;
detecting a multi-feature query of the database; and
generating a multi-feature visualization based on the multi-feature query.

2. The method of claim 1 further comprising importing data regarding a plurality of features for a plurality of census-defined places, counties, and states.

3. The method of claim 1 wherein the one or more geographically defined areas comprise latitude and longitude coordinates.

4. The method of claim 1 wherein the polygons comprise points and vectors associated with specific municipally-defined areas.

5. The method of claim 1 further comprising defining the one or more geographically defined places as a plurality of polygons based on Topologically Integrated Geographic Encoding and Referencing system (TIGER) data.

6. The method of claim 1 wherein the one or more geographic levels comprise one or more of a census tract, a census-defined place, a county, a collection of counties, a state, and a user-defined geography.

7. The method of claim 1 wherein the population health data comprises cancer data by population.

8. The method of claim 7 wherein the population health data comprises cancer data from at least one of the Florida Department of Health, the Florida Cancer Data System, the Florida Stroke Registry, and the Behavioral Risk Factor Surveillance System.

9. The method of claim 1 wherein the population health data comprises stroke data by population.

10. A non-transitory computer-readable medium comprising instructions that when executed, cause one or more processors to perform the steps of claim 1.

11. A system for displaying a dynamic, multi-feature representation of health data, based on aggregated data from multiple sources, the system comprising:
a database configured to store data regarding a plurality of features related to health data; and
one or more processors communicatively coupled to the database and configured to import data regarding a plurality of features for a plurality of census tracts to the
database;
define a plurality of geographically defined areas as polygons with associated labels;
overlay the plurality of census tracts on the polygons;
associate census tracts falling within a polygon to a geographically defined area defined by the polygon;
perform a best fit for each census tract that crosses a boundary of the one or more geographically defined areas;
associate census tracts with the one or more geographically defined areas based on the best fit;
for each of the plurality of geographically defined areas, aggregate the census tract data for each feature based on the associating;
receive population health data at one or more geographic levels;
associate the population health data by geographic level to the corresponding one or more geographically defined areas;
receive a multi-feature query of the database; and
generate a multi-feature visualization based on the multi-feature query.

12. The system of claim 11 wherein the one or more processors are further configured to import data regarding a plurality of features for a plurality of census-defined places, counties, and states.

13. The system of claim 11 wherein the one or more geographically defined areas comprise latitude and longitude coordinates.

14. The system of claim 11 wherein the polygons comprise points and vectors associated with specific municipally-defined areas.

15. The system of claim 11 wherein the one or more processors are further configured to define the one or more geographically defined places as a plurality of polygons based on Topologically Integrated Geographic Encoding and Referencing system (TIGER) data.

16. The system of claim 11 wherein the one or more geographic levels comprise one or more of a census tract, a census-defined place, a county, a collection of counties, a state, and a user-defined geography.

17. The system of claim 11 wherein the population health data comprises at least one of cancer data and stroke data by population.

18. The system of claim 17 wherein the population health data comprises cancer data from at least one of the Florida Department of Health, the Florida Cancer Data System, the Florida Stroke Registry, and the Behavioral Risk Factor Surveillance System.

19. A computer-implemented method for displaying a dynamic, multi-feature representation of health data, based on aggregated data from multiple sources, the method comprising:
importing, by one or more processors, data regarding a plurality of features for a plurality of municipal cells to a database;
defining a plurality of geographically defined areas as polygons with labels;
overlaying the plurality of municipal cells on the polygons;
associating municipal cells falling within a polygon to a geographically defined area defined by the polygon;
performing a best fit for each municipal cells that crosses a boundary of the plurality of geographically defined areas;
associating municipal cells with the plurality of geographically defined areas based on the best fit;
for each of the plurality of geographically defined areas, aggregating the municipal cell data for each feature based on the associating;
receiving population health data at one or more geographic levels;
associating the population health data by geographic level to the corresponding geographically defined area;
detecting, by the one or more processors, a multi-feature query of the database; and
generating, by the one or more processors, a multi-feature visualization based on the multi-feature query.

20. The method of claim 19 wherein the municipal cells comprise one or more of census-defined places, counties, and states.

21. The method of claim 19 wherein the one or more geographic levels comprise one or more of a census tract, a census-defined place, a county, a collection of counties, a state, and a user-defined geography.

22. A non-transitory computer-readable medium comprising instructions that when executed, cause one or more processors to perform the steps of claim 19.
